(19) United States
(12) Patent Application Publication
(10) Pub. No.: US 2003/0113823 A1
(43) Pub. Date: Jun. 19, 2003

(54) Diagnostic assays for determination of dental caries susceptibility

(76) Inventor: Richard L. Gregory, Carmel, IN (US)

Correspondence Address: FULLBRIGHT & JAWORSKI L.L.P., 600 CONGRESS AVE., SUITE 2400, AUSTIN, TX, 78701, US

(21) Appl. No.: 10/268,017

(22) Filed: Oct. 9, 2002

Related U.S. Application Data
(60) Provisional application No. 60/328,537, filed on Oct. 11, 2001.

(51) Int. Cl. G01N 33/554; G01N 33/569; A61K 7/16

(52) U.S. Cl. 435/7.32; 424/49

(57) ABSTRACT

The invention overcomes the limitations of the prior art by providing rapid assays for predicting the likelihood of caries development in patients. The assays allow implementation of appropriate dental care measures during a patient visit depending on the results of the assay. The assay utilizes the finding that caries-free children and adults have significantly higher levels of naturally occurring protective salivary IgA antibody to S. mutans than caries-active subjects. The assays are carried out using patient saliva. The speed and ease of use of the assay allows dental practitioners to assess at an early stage the relative risk of future caries formation. With this information, preventive methods may be applied only to those determined to be at risk.

This application claims the priority of U.S. Provisional Patent Application Ser. No. 60/328,537, filed Oct. 11, 2001. The government may own rights in the present invention pursuant to grant number DE007125-20 from the National Institute of Dental and Cranialfacial Research.

### BACKGROUND OF THE INVENTION

1. Field of the Invention

The present invention relates generally to the field of dentistry. More particularly, it concerns assays for the identification of individuals susceptible to future caries development.

2. Description of Related Art

Streptococcus mutans has been established to be the main etiologic factor in the development of dental caries (Loesche, 1986). Like other bacterial cells, S. mutans has surface antigens which are unique and enable the cell to adhere to the smooth surfaces of teeth. The most important cell attachment antigens include glucosyltransferase, antigen I/II, and fimbriae present on the cell surface. Glucosyltranferase (GTF) is a complex of enzymes on the cell surface which are responsible for adherence of the cell to enamel through a mechanism which involves cleaving sucrose into insoluble and soluble glucose polymers called glucans. These glucans bind to the pellicle on the tooth, enabling the cell to attach to the tooth surface. In addition, the glucans serve to promote inter-bacterial binding. Antigen I/II is present on the surface of the bacterial cell and promotes binding of the cell to the tooth surface (reviewed in Gregory, 1994a). Fimbriae are small hairlike appendages which extend from the cell surface, allowing the cell to adhere to pellicle-coated tooth surfaces in a sucrose-independent manner.

One of the body's defenses against S. mutans is secretory IgA antibodies. Secretory IgA is present in saliva, tears, breast milk, and secretions bathing the lamina propriae of the gastrointestinal, respiratory, and genitourinary tracts. Since S. mutans is a normal inhabitant of the oral cavity, it is swallowed and allowed to enter the digestive tract. Peyer's patches, specialized immune tissues, are present in the small intestine. Presentation of S. mutans antigens to the Peyer's patches induces activated B and T lymphocytes to leave and travel through the circulation to mucosal surfaces of the body, where they differentiate into plasma cells and secrete specific secretory IgA antibodies to the S. mutans antigen (reviewed in Gregory, 1994b).

S. mutans has virulence factors that enable the bacterium to multiply, adhere to smooth surfaces and produce organic acids. These properties are dependent on specific enzymes present on the cell surface, specifically GTF and phosphotransferase (PTS). Some of the secretory IgA antibodies that are secreted are specific to these S. mutans enzymes and have been shown to be effective in neutralizing GTF and PTS, thus inhibiting the more complex virulence factors such as growth, acid production and adherence. Neutralization of GTF would lead to decreased adherence and neutralization of PTS should decrease growth and all other metabolic activities of the cell. In addition, binding of specific secretory IgA antibodies to antigen I/II and fimbriae have been demonstrated to inhibit S. mutans colonization. Gregory, et al. (1990) and Fontana et al. (1995) and others have established that individuals who have higher levels of specific secretory IgA to S. mutans antigens (including GTF, antigen I/II, and fimbriae) are much more caries resistant than individuals who have lower levels of secretory IgA antibody.

Although the prior art has furthered the understanding of caries development, there still remains no diagnostic tool to rapidly predict future caries activity with accuracy. Existing laboratory assays allow specific determination of antibody levels, yet no chairside assays has been developed for use while the patient is available for preventive measures. The ability to rapidly assay a patient for likelihood of caries development would allow implementation of appropriate measures during in-office visits with patients, increasing the cost-effectiveness and convenience of treatment. There is, therefore, a great need in the art for rapid assays predictive of caries development activity.

### SUMMARY OF THE INVENTION

In one aspect, the invention provides a diagnostic apparatus comprising: (a) a porous solid support; (b) microparticles reversibly attached to said support, wherein said microparticles are bound to at least a first antigen from Streptococcus mutans and wherein the microparticles are capable of migrating along the support when contacted with human saliva; and (c) a ligand bound to said support, wherein said ligand has an affinity for at least a first human IgA antibody. Any suitable solid support may be used, including, for example, nitrocellulose. The microparticles may be comprised of any suitable material, including, for example, latex, and the microparticles may be epoxy modified. It will be understood to those of skill in the art that the term “microparticles” encompasses particles of any size that are capable of diffusing in or being suspended in saliva with the assays of the invention. Beads are a type of microparticle.

The microparticles may be bound to a plurality of antigens from Streptococcus mutans. The antigens may be substantially purified and may also comprise a crude fraction of antigens. Examples of antigens that could be used include glucosyltransferase, Antigen I/II, and a fimbrial protein, such as SmaA. Any suitable method may be used for detection of the microparticles. For example, the beads may be colored and may be labeled. Suitable labels may, for example, be visually detectable, such as a fluorescent label. The microparticles could also be labeled with a second antigen or enzyme. The microparticles may latex beads. The beads may, for example, average from about 0.15 μm to about 0.3 μm in diameter. Preferably, the microparticles are reversibly bound at a first selected location on said solid support and wherein the ligand is bound at a second location on said solid support.

The saliva used for assays may or may not be diluted. Exemplary dilutions include about 1:2 to about 1:100 in water, including 1:2 and 1:3. It will be understood that the term “water” specifically includes aqueous solutions containing various buffers or other ingredients.

A ligand used with the assays may be a protein, such as a binding protein, and may be and antibody or fragment thereof. In one embodiment of the invention, the ligand binds specifically to a human IgA Fc region. At least a second ligand may also be bound to said support. The ligand may be an antibody or any other type of ligand. In one embodiment, second ligands are used as controls for saliva migration, bead migration and/or the presence of IgA. For example, the ligand may have an affinity for a protein present in saliva, including amylase. The second ligand may also have affinity for at least a first human IgM antibody or a first antigen from Streptococcus mutans.

In another aspect, the invention provides a method of assaying a patient for susceptibility to caries development comprising the steps of: (a) obtaining an assay apparatus comprising: (1) a porous solid support; (2) microparticles reversibly attached to said support, wherein said microparticles are bound to at least a first antigen from Streptococcus mutans and wherein the microparticles are capable of migrating along the support when contacted with a solution comprising human saliva; and (3) a ligand bound to said support at a selected location, wherein said ligand has an affinity for a human IgA antibody; (b) contacting said assay apparatus with saliva from a patient, wherein said saliva is allowed to contact said microparticles and said ligand; and (c) detecting the presence or absence of microparticles bound to said ligand at said selected location.

The apparatus used for the method may comprise any suitable solid support, including, for example, nitrocellulose. The microparticles may be comprised of any suitable material, including, for example, latex, and the microparticles may be epoxy modified. It will be understood to those of skill in the art that the term “microparticles” encompasses particles of any size that are capable of diffusing in or being suspended in saliva with the assays of the invention. Beads are a type of microparticle. The microparticles may be bound to a plurality of antigens from Streptococcus mutans. The antigens may be substantially purified and may also comprise a crude fraction of antigens. Examples of antigens that could be used include glucosyltransferase, Antigen I/II, and a fimbrial protein, such as SmaA. Any suitable method may be used for detecting the microparticles. For example, the beads may be colored and/or may be labeled. Suitable labels may, for example, be visually detectable, such as a fluorescent label. The microparticles could also be labeled with a second antigen or enzyme. The microparticles may be latex beads, which may, for example, average from about 0.15 μm to about 0.3 μm in diameter. Preferably, the microparticles are reversibly bound at a first selected location on said solid support and wherein the ligand is bound at a second location on said solid support.

The saliva used in the method may or may not be diluted. Exemplary dilutions include about 1:2 to about 1:100 in water, including 1:2 and 1:3. It will be understood that the term “water” specifically includes aqueous solutions containing any desired buffers or other ingredients, whether added or not. A ligand used with the assays may be a protein, such as a binding protein, and may be and antibody or fragment thereof. In one embodiment of the invention, the ligand binds specifically to a human IgA Fc region. At least a second ligand may also be bound to said support. The ligand may be an antibody or any other type of ligand. In one embodiment, second ligands are used as controls for saliva migration, bead migration and/or the presence of IgA. For example, the ligand may have an affinity for a protein present in saliva, including amylase. The second ligand may also have affinity for at least a first human IgM antibody or a first antigen from Streptococcus mutans.

In still yet another aspect, the invention provides a method of dental care comprising: (a) obtaining saliva from a patient; (b) rapidly assaying said saliva for an IgA antibody specific to an antigen from Streptococcus mutans while the patient waits, wherein results from said assaying are obtained within 30 minutes from said step of obtaining; and (c) initiating a course of treatment for prevention of caries development based on said rapid assaying. The rapidly assaying may comprise, for example, the steps of: (a) obtaining an assay apparatus comprising: (1) a porous solid support; (2) microparticles reversibly attached to said support, wherein said microparticles are bound to at least a first antigen from Streptococcus mutans and wherein the microparticles are capable of migrating along the support when contacted with a solution comprising human saliva; and (3) a ligand bound to said support at a selected location, wherein said ligand has an affinity for a human IgA antibody; (b) contacting said assay apparatus with saliva from a patient, wherein said saliva is allowed to contact said microparticles and said ligand; and (c) detecting the presence or absence of microparticles bound to said ligand at said selected location. The course of treatment may comprise a preventive measure selected from the group consisting of: recommending immaculate oral hygiene, recommending diet modification, applying a sealant, applying an antimicrobial agents, applying a topical fluoride treatments and applying a fluoride varnish.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Cross-section of typical immunochromatographic strip unit. Components of membrane strip: 1) Sample pad; 2) Conjugate pad; 3) Hi-Flow membrane; 4) Absorbent pad; 5) Laminate card; and 6) Top laminate

FIGS. 2A-C. Examples of immunochromatographic strip assay procedures. FIG. 2A: General process of immunochromatographic strip exposed to saliva adapted from Bang's Laboratories, TechNote #204 Adsorption to Microspheres, and TechNote #303 Lateral Flow Tests, 1999. (1) Diluted Saliva, (2) End to be dipped in saliva, (3) Band of blue latex beads coated with S. mutans antigen (initial placement), (4) Anti-human IgA antibody line. FIG. 2B. Diagram of immunochromatographic strip exposed to saliva from a caries-free subject containing high levels of anti-S. mutans antibodies indicating a blue band at the site of the anti-human IgA band; (1) Diluted Caries-Free Saliva, (2) End to be dipped in saliva, (3) Band of blue latex beads coated with S. mutans antigen (initial placement), (4) Anti-human IgA antibody line. FIG. 2C. Diagram of immunochromatographic strip exposed to saliva from a caries-active subject containing low levels of anti-S. mutans antibodies (or a saline negative control) indicating a lack of a blue band at the site of the anti-human IgA band. Instead the blue latex bead band will have migrated to the end of the strip; (1)Diluted Caries-Active Saliva or Saline, (2) End to be dipped in saliva, (3) Band of blue latex beads coated with S. mutans antigen (initial placement), (4) Anti-human IgA antibody line.

FIG. 3. Latex bead strip assay demonstrating migration and binding of the blue antigen-coated beads to the anti-human IgA line on the membrane with saliva. Arrow indicates strong band of latex beads coated with S. mutans antigen and salivary IgA antibodies immobilized by anti-human IgA after dipping in saliva. (1) indicates location of latex bead application, (2) indicates the application site of the anti-human IgA antibody line.

FIG. 4. Latex bead strip assays demonstrating migration and binding of the blue antigen-coated beads to the anti-human IgA line and formation of a blue band on the membrane with saliva (A) or migration of beads and lack of a band after dipping in saline (B). Arrows indicate the application site of the anti-human IgA antibody line.

FIG. 5. Latex bead strip assays demonstrating migration and binding of the blue antigen-coated beads to the anti-human IgA line on the membrane with saliva (1:3 dilution on right hand strip and/or 1:10 dilution on strip at left) from 5 normal subjects. Subject 1 only had 1:3 dilution of saliva and subjects 2-5 had both dilutions. Arrow indicates the application site of the anti-human IgA antibody line.

### DESCRIPTION OF ILLUSTRATIVE EMBODIMENTS

The invention overcomes the limitations of the prior art by providing rapid assays for predicting the likelihood of caries development in patients. The assays allow implementation of appropriate dental care measures during a patient visit depending on the results of the assay. The results of the inventor's studies demonstrate that the diagnostic assays can rapidly determine the presence of salivary IgA antibody. The assays have important implications for the implementation of novel in-office diagnostic techniques for the direction of medical care.

The assays provided by the invention utilize the finding that caries-free children and adults have significantly higher levels of naturally occurring protective salivary IgA antibody to S. mutans than caries-active subjects. The assays are carried out using patient saliva. The speed and ease of use of the assay allows dental practitioners to assess at an early stage the relative risk of future caries formation. With this information, preventive methods may be applied only to those determined to be at risk.

In one embodiment of the invention, the assay comprises a diagnostic reagent strip that detects secretory IgA antibodies to S. mutans in saliva from patients. The diagnostic reagent strip can be detected if a colored line (for example, blue) appears at the appropriate location following application of saliva from a patient with high salivary secretory IgA antibody levels. This indicates that the patient has no significant risk of developing dental caries and further intervention for prevention of caries development is not needed. In contrast, patients that will develop future caries will not have a blue or other colored line appear at that location on the strip when their saliva is tested.

In a preferred embodiment of the invention, the assay is prepared by attaching S. mutans surface antigens to blue latex beads, for example, carboxyl modified (0.2 μm and 0.289 μm) and epoxy modified (size 0.360 μm) beads. In certain embodiments of the invention, use of 0.2 μm carboxyl modified beads may be preferred. The beads, or any other appropriate microparticles, can be placed on an appropriate support, such as a nitrocellulose membrane. The microparticles are attached in such a manner that upon contact with saliva, the beads can migrate along the support. When the strip of support material is dipped in saliva, the saliva moves up the strip by capillary action. When the saliva reaches the microparticles coated with S. mutans antigen, any S. mutans-specific secretory IgA binds to the antigens. The microparticles move up the paper where they meet a fixed band of anti-human IgA, or another ligand, that has also been attached to the support. An antigen-antibody reaction occurs immobilizing the microparticles containing salivary secretory IgA anti-S. mutans antibodies, thus causing a colored line to develop on the support material when colored beads are used. Alternatively, any other known technique may be used for detection of the microparticles, such as use of a label.

The saliva used for assays may be obtained directly from a patient. However, it was found that the antibody detection reaction could be optimized by use of saliva dilutions, such as diluting the saliva sample 1:3. Similarly, dilution of anti-human IgA 1:2 obtained from the manufacturer was preferable. When using the 1:3 dilution of saliva, it was found to take a front of latex beads approximately 120 seconds to travel 4.0 cm with a nitrocellulose membrane. In contrast, when the strip is dipped in water, the beads move up the membrane but are not bound by the anti-human IgA.

Internal controls for saliva and bead migration and IgA deficiency may be included with the assays. For example, a saliva migration control may be used, comprising a line of anti-amylase antibody or antibodies of another salivary protein placed on the support past the anti-human IgA antibody line described above relative to a location where the support is contacted with saliva. Differentially labeled beads, for example, red beads coated with the same anti-amylase antibody may be mixed with the labeled S. mutans antigen-coated beads and placed at the same or a proximal location. As amylase is a key component of human saliva and is found in all normal saliva samples, the amylase in saliva will be bound by the anti-amylase on the red beads and carried by capillary action to the anti-amylase line on the strip, where it will remain bound. This will thus serve as a control to assess the ability of saliva to migrate on the support strip. The absence of a red or otherwise differentially labeled band located at the location of the anti-amylase will indicate the lack of a suitable amount of saliva implying a possible dilution or other error.

A bead migration control may also be used, for example, comprising a line of anti-S. mutans antibody placed on the strip past the anti-human IgA and anti-amylase lines, relative to the location where the support is contacted with saliva. Some of the same beads coated with S. mutans antigen will migrate past the anti-human IgA and anti-amylase lines because they do not contain a suitable amount of salivary IgA antibody. Capillary action of the saliva will carry the blue beads (or otherwise detectable) to the anti-S. mutans line on the strip where it will remain bound. This control will assess the ability of the beads to migrate on the strip. The absence of bound beads at the anti-S. mutans antibody location will indicate the lack of bead migration. In a properly working system using controls, there should be both of the differentially detectable bands (for example, red and blue) located at the anti-amylase and anti-S. mutans lines. Lack of these bands will indicate an abnormal result.

It should be noted that IgA deficiency affects between 1/200 and 1/1000 individuals, with a substantial subset of these individuals compensating with secretory IgM antibodies to S. mutans in saliva. IgA deficient individuals without compensating IgM antibody to S. mutans have significantly higher levels of caries than normal controls. However, IgA deficient subjects with compensating IgM have normal caries rates. An IgA deficiency control may therefore be placed on the opposite side of the strip which will have similar components (i.e., absorbent and samples pads, and nitrocellulose membrane). The IgA deficient control will preferably be composed of a mixture of red beads coated with anti-human IgA antibody and blue beads coated with the same S. mutans antigen as placed on an opposing side of the support.

Anti-human IgM and anti-human IgA antibody lines are preferably placed at the opposite end of the support strip from the sample application site. Normal saliva samples containing suitable concentrations of IgA will be bound to the beads coated with anti-IgA and migrate to the anti-IgA line and remain there, where they will be detectable. Saliva samples containing low IgA concentrations (i.e., IgA deficient) will not form the detectable line at this site. Saliva samples containing a large amount of secretory IgM antibody to S. mutans antigens will bind to S. mutans-coated beads (for example, blue beads) and migrate to the anti-IgM line and remain there, where they will be detectable. This will indicate an individual that is an IgM compensating individual, and if the subject also has a low concentration of IgA, this subject may be able to compensate for the lack of IgA and remain immunologically protected from caries and not need additional preventive dental procedures. However, if the saliva from this subject does not have detectable beads on this side of the strip, then that individual has an IgA deficiency without IgM compensation and will require preventive dental procedures.

### I. RATIONALE AND SIGNIFICANCE OF THE INVENTION

The great majority of adults carry significant numbers of immunogenic S. mutans in dental plaque and saliva. S. mutans has at least 3 colonization factors/mechanisms that are responsible for adherence to the tooth surface, including two sucrose-independent protein-binding ligands (antigen I/II and fimbriae) and one sucrose-dependent adherence mechanism (glucosyltransferase). Caries-free children and adults have significantly higher levels of naturally occurring protective salivary secretory IgA antibody to each of the three factors that inhibit their activity than do caries-active subjects. Furthermore, human volunteers and experimental animals can be successfully immunized mucosally with S. mutans antigens leading to increased specific protective secretory IgA antibody which is associated with reduced numbers of plaque and salivary S. mutans and reduced carious lesions.

Unfortunately, there have been no diagnostic tools with the sensitivity or specificity available clinically to allow the prediction of future caries activity. Laboratory-based assays have existed for many years that would allow the specific determination of antibody levels, but no assays have been developed for chairside use while the patient is available to institute preventive measures.

A rapid diagnostic assay utilizing saliva from dental patients, primarily focusing on the pediatric population, would be of great benefit to the patient. Development of this assay would allow the dental practitioner to assess at an early stage the relative risk of future caries formation in a particular individual.

With this information, caries preventive methods may be applied to those determined to be at risk, while those patients not at risk may not be recommended for the same level of preventive measures. For example, if a patient is determined to be susceptible to future caries development, this could be explained to the patient and preventive measures provided, such as a recommendation for immaculate oral hygiene, diet modification, increase in salivary flow modifications (i.e., change in medication for hypertension), sealants, chlorhexidine or other antimicrobial agents, topical fluoride treatments or fluoride varnishes. These measures could be applied either locally at standard high risk sites (deep fissures) or provided to the whole mouth.

Prevention of caries remains an extremely cost effective means compared with restoring carious lesions. In addition, the ability to determine caries at risk patients allows only those at risk to receive the preventive treatment and not the entire population. Overall, the costs of providing dental care for both types of patients should be significantly decreased.

### A. Background and Significance of the Invention

S. mutans from human carious lesions was first reported in 1924 by Clarke who suggested that the acidogenic and aciduric properties of this organism may be responsible for dental decay. However, it was not until 1960 when Fitzgerald and Keyes demonstrated the infectious and transmissible nature of oral streptococci, that research on the bacterial etiology of caries was continued. S. mutans has since been associated with natural dental caries in many species including monkeys (Bowen, 1969), rats (Michalek et al., 1975) and hamsters (Hamada et al., 1978). There is a definite association of the presence of S. mutans in the human oral cavity with dental caries (for review, see McGhee and Michalek, 1981) and this organism is now considered to be the major causative agent of human dental caries (Loesche and Straffon, 1979).

### B. Role of Surface Antigens in Attachment

Sucrose-independent and dependent adhesins for S. mutans have been identified. Surface antigen I/II (sAg I/II) also known as P1, and fimbrial proteins have been demonstrated to play important roles in sucrose-independent colonization of S. mutans to saliva-coated surfaces. Glucosyltransferase (GTF) has also been established through its sucrose-dependent ability to synthesize large amounts of insoluble glucans that serve to bind bacteria carrying glucan-binding proteins (GBP) to other bacteria containing GBP or GTF. GTF, a complex of S. mutans surface proteins, of molecular weight approximately 157-200 kDa, is involved in the synthesis of soluble and insoluble glucans which provide sucrose-dependent attachment to the salivary pellicle. Glucans are produced by metabolism of sucrose to a polysaccharide. GBPs have been described in several of the oral streptococci. S. mutans has two GBP with glucan-binding capabilities (59 and 74 kDa). S. mutans serotype c organisms express a protein of approximately 185 kDa designated by a number of laboratories as either sAgI/II (Russell, M. W. et al., 1980; Zanders and Lehner, 1981) or P1 ; (Forester et al., 1983), which mediates attachment to saliva-coated hydroxyapatite (HA) (Lee et al., 1989; Koga et al., 1990). P1 is thought to be associated with fibrils on S. mutans surface (Ayakawa et al., 1987). The P1-like proteins are believed to function as adhesins in vivo enabling S. mutans to bind to the salivary pellicle on teeth and to other plaque bacteria (McBride et al., 1984).

It has been reported that fimbriae from S. mutans isolates obtained from caries susceptible subjects had a greater proportion of GTF, P1 and fimbrial proteins than the fimbriae of S. mutans isolates obtained from caries resistant subjects (Perrone et al., 1997). Another important finding in this study was the presence of a 65 kDa fimbrial protein in all the isolates but in greater relative proportions in the fimbriae from caries susceptible subjects. In a typical model, the fimbriae serve as a ligand and the receptor would most likely be a component on the tooth surface, probably amylase. The tooth is bathed and coated in saliva, and therefore saliva serves as a receptor for the fimbriae of S. mutans. The 65 kDa fimbrial protein binds to amylase and has been termed S. mutans adhesin A (SmaA) by this laboratory (Ray et al., 1999). SmaA has been observed in all S. mutans isolates examined, suggesting that it may be conserved in this species.

### C. Role of Salivary Components in Attachment to Oral Streptococci

Saliva is a very heterogeneous mixture of proteins that have various functions. Saliva serves as a primary host component for metabolism, host defense, and also a receptor for bacterial attachment to host tissues (Levine et al., 1978). The proteins associated with attachment include proline-rich peptides (PRP), histidine-rich peptides, secretory IgA, lactoferrin, statherin, salivary mucins, amylase and several proteins with molecular weights 55 and 60 kDa. Data further suggest that S. mutans may bind to amylase-coated surfaces through fimbrial receptors (Ray et al., 1999).

### D. Colonization Mechanisms of S. mutans

One of several proposed mechanisms of the adherence of S. mutans to enamel surfaces is summarized below (for reviews, see McGhee and Michalek, 1981; Hamada et al, 1986). Rolla (1977) has suggested that the interaction between negatively charged oral bacteria and positively charged HA surfaces may serve as one of the first reversible steps in the adherence of S. mutans to enamel surfaces. HA is a crystalline lattice composed of calcium, hydroxyl and orthophosphate and is a component of enamel. Cell to cell adherence is important in the formation of dental plaque. Many strains of S. mutans form aggregates with other oral bacteria upon addition of glucan (Gibbons and Fitzgerald, 1969; Gibbons and Van Houte, 1980), thus dental plaque can grow in size by permitting oral bacteria to adhere to glucan produced primarily by S. sanguis or Actinomyces in vivo. This agglutinating activity is independent of S. mutans GTF activity and indicates the complexity of the cell to cell and cell to pellicle adherence mechanism of S. mutans (reviewed by Gibbons and Van Houte, 1980).

It appears that the adherence of S. mutans and other oral bacteria to pellicle-covered enamel occurs in two steps. The initial attachment of single cells, chains of cells or aggregated cells may involve the negative charges on the bacterial cells, divalent cations and positive charges in the pellicle including sucrose-independent functions such as sAgI/II and fimbriae. The second phase of the process, or maturation of the plaque, appears to depend mostly on the multiplication of S. mutans and synthesis of insoluble glucan by other bacteria. Bacteria entering the developing plaque after the initial attachment phase may do so both by binding to specific receptor sites and/or random contact with the adhesive glucan of the plaque. Many of these bacteria also have glucan receptors on their cell surface.

### E. Natural Immunity to Caries

The principal immunoglobulin in external secretions is secretory immunoglobulin A (secretory IgA) and its role in protection against certain mucosal pathogens has been established (McGhee and Mestecky, 1983; Mestecky, 1988; Mestecky et al., 1986). Secretory IgA antibodies have been found to inhibit microbial adherence, colonization and penetration of mucosal surfaces, inhibit metabolic pathways, neutralize enzymes, viruses and toxins, mediate expulsion of plasmids, agglutination of microbes and inhibit the growth of certain organisms (Bellanti et al., 1965; Walker et al., 1972; Michalek et al., 1976; Pierce et al., 1982; Gregory et al., 1984; McGhee and Mestecky, 1983). Naturally occurring salivary IgA antibodies to S. mutans are present in most individuals and first appear early in childhood as a result of swallowed S. mutans antigens being processed by the common mucosal immune system. Camling and Kohler (1987) reported that salivary IgA antibody to S. mutans appears between the ages of 1 and 5 years. Smith et al. (1990) reported in a longitudinal study that the IgA antibody level to S. mutans continues to increase from 5 months to 3 years of age and Gahnberg et al. (1985) confirmed these findings in the age group from 2 to 48 months of age. Caries-free adults and children have been reported to have significantly higher levels of naturally occurring salivary IgA and serum antibodies to S. mutans than caries-active subjects (Bammann and Gibbons, 1979; Camling et al., 1987; Aaltonen et al., 1987). Specific salivary antibodies to S. mutans inhibit adherence and acid production and other enzyme activities of S. mutans (Germaine and Tellefson, 1981; Gahnberg et al., 1985; Gregory et al., 1990). In addition, Lehner and colleagues have consistently demonstrated negative correlations between caries levels and serum antibody titers to S. mutans (Challacombe and Lehner, 1976; Lehner et al., 1978).

While the majority of the human population develop dental caries, some individuals remain caries-free throughout their lives. Every individual is immunologically exposed to virulence determinants on the surface of this organism by ingesting up to 10¹¹ S. mutans cells/day. Orally immunized humans develop high levels of secretory IgA antibodies to S. mutans in saliva (Mestecky et al., 1978; Czerkinsky et al., 1987; Gregory and Filler, 1987). Caries-resistant (CR) individuals and immunized animals produce salivary antibodies that protect them from this disease, while the majority of the population and unimmunized control animals do not. Studies have shown that naturally occurring and induced salivary IgA antibodies from adults and experimental animals inhibit S. mutans colonization and adherence, GTF and glucose-PTS activity (McGhee et al., 1975; Michalek et al., 1976; Taubman and Smith, 1976; Gregory et al., 1986; Gregory et al., 1990). Preliminary studies using induced antibodies from experimental animals immunized with S. mutans whole cells or purified antigens inhibited growth, acid production, glucose-PTS and glucose uptake. These inhibitory activities may explain the natural S. mutans-inhibitory state in caries-free adults.

However, the protective functional aspects of naturally occurring salivary IgA antibodies from caries-free (CF) and caries-susceptible (CS) children to virulence factors has not been precisely examined. CR adults have significantly higher levels of binding salivary IgA and serum IgG antibodies to S. mutans whole cells, serotype CHO, GTF, sAgI/II and fimbriae than CS adults by ELISA (Gregory et al., 1995; Fontana et al., 1995). In addition, CF children have significantly greater levels of binding salivary IgA antibody to S. mutans than CS children by ELISA (Rose, et al., 1994). These results indicate an important role for naturally occurring salivary IgA and serum IgG antibodies in regulation of S. mutans virulence factors and human dental caries.

### II. RAPID DIAGNOSTIC ASSAYS

One important embodiment of the invention provides diagnostic assays comprising lateral flow strip tests. The advantages of the assay include user-friendly format, very short time to get results, long term stability over a wide range of climates, and relatively inexpensive production. These features make the test ideal for rapid point of care testing. However, lateral flow strip assays have not been available for the diagnosis of any dental disease.

The sensitivity of tests developed for non-dental applications has been estimated at 0.1-0.2 fmol of antigen or antibody/ml, making these assays comparable or exceeding the sensitivity of reference laboratory-based ELISA assays. Although all assays to date are qualitative, methods are being developed to quantitate the data. Strips of support materials are porous in nature, allowing test fluids to flow through the strip. A preferred material for assays are membranes (commonly nitrocellulose) which have tiny pores, permitting movement of fluid and latex beads through the strip or along the surface of the membrane. Researchers can attach a variety of components to the nitrocellulose membrane, including antibodies and proteins that react with the substance of interest. Another component that is typically added to the strip are colored latex beads which have either antibody or protein antigen attached to the surface. Once all components are attached to the membrane, the body fluid is applied which moves through the strip by capillary action. If the substance of interest is present, a chemical reaction will take place and a certain colored line of beads will appear in a known location on the strip.

The presence of a given antibody will usually be measured on the basis of their presence/absence (yes/no). For immunometric-type assays, a ligand specific for the analyte (normally, but not necessarily an antibody [Ab]) is immobilized to the membrane. The detector reagent, typically an antibody coupled to latex beads or colloidal metal, is deposited (but remains unbound) into the conjugate pad. When sample saliva is added to the sample pad, it rapidly wets through to the conjugate pad and the detector reagent is solubilized. The detector reagent begins to move with the sample flow front up the membrane strip. Analyte that is present in the sample will be bound by the antibody that is coupled to the detector reagent. As the sample passes over the zone to which a capture ligand has been immobilized, the analyte detector reagent complex is trapped. Color develops in proportion to the amount of analyte present in the sample.

Since secretory IgA antibody levels to S. mutans have been proven to be associated with caries resistance or susceptibility, a test that can predict caries activity in individual patients has been created. Higher levels of secretory IgA antibody are associated with caries resistance, while lower levels are associated with caries susceptibility. We will be using saliva samples from children who will be classified as caries-free or caries-active. Using this diagnostic reagent strip, which will be dipped into the saliva from these individuals, we predict that individuals who are caries-free will have a blue line appear on the strip, indicating that they have high levels of secretory IgA. Likewise, we predict that saliva from caries-active individuals will not produce a blue line on the strip, indicating lower levels of secretory IgA.

### III. ASSAY DESIGN AND METHODS

### A. Patient Population Demographics for Large Scale Human Trials

The choice of human subjects for proposed large scale studies is of central importance. The studies may be used to investigate the association of salivary IgA antibodies to S. mutans with caries activity using the rapid immunological diagnostic caries susceptibility assay. For this, it is imperative that the correct populations be selected. In this regard, healthy CF and CS subjects, between the ages of ten and thirteen years of age, are selected from the patient population of the Oral Health Research Institute at Indiana University on the IUPUI Campus by Dr. Dominick Zero and staff. This patient population includes approximately 1000 children in the Oral Health Research Institute data base. Approximately 250 children are initially recruited for this project as part of an ongoing study based in Connersville, Ind.

Whole saliva is collected from all subjects and stored on ice for no more than 5 h until frozen at −20° C. All samples are assayed within 2 weeks. Included in these samples are whole saliva samples collected from qualifying CF and CS subjects. At least 20 subjects/group are recruited for a total of 40 subjects. No subjects are recruited with a history of antibiotic usage within the past 4 weeks. The CF group is defined as those without carious lesions or restorations. Systemically healthy CS controls are also elected. CS status is defined as those individuals having had five or more carious lesions (DMFT). A follow up exam is conducted after 12 months to validate either CF or CS status. CF subjects should not have any additional carious lesions. Many CS subjects should have increased numbers of affected surfaces. Only CS subjects that develop at least two additional lesions (DMFT) in the intervening 12 months are validated as true CS subjects. Likewise, only CF subjects that remain free of caries during the 12 month period are termed true CF subjects. Age, sex and race matched controls are used in all cases (one CS/CF subject). Approximately half of the subjects are female and half are male.

It is anticipated that within the 250 member pediatric subject population it will be possible to identify at least 20 CF and 20 CS subjects for use in the study. It is expected that some of the initial CF children will develop carious lesions during the 12 month follow-up interval and some of the CS subjects will not develop additional lesions. All samples are assessed using both ELISA and the rapid diagnostic assay, however, determining sensitivity and specificity of the diagnostic assay will not be the major focus of this study.

### B. Clinical Determination of Caries Status

Detection of dental caries is accomplished with an intraoral examination including the use of a fine tipped dental explorer and dental operatory lighting by the criteria of Radike et al. (1973) and bite-wing radiographs when indicated. DMFT scores are determined for each subject. One age-, sex- and race-matched CS subject is recruited for each CF child. Previously, no significant difference in secretory IgA antibody levels was found between CF adult subjects receiving fluoride and those CF adults having a minimal history of fluoride usage (Gregory et al., 1990). The same was true for CS adults. Therefore, no stratification is done to control for fluoride history. The flow rate of whole saliva is correlated with both DMFT and antibody levels using both the rapid diagnostic assay and ELISA to attempt to establish a connection between clinical appearance and antibody levels. Each subject is instructed to not eat or brush for at least 1 h, or use mouthwash for 12 h prior to appointment to standardize collection procedures.

### C. Saliva Collection

All CF and CS volunteers are asked to donate whole saliva. Unstimulated whole saliva is collected by expectoration into a graduated 50 ml centrifuge tube over either a 5 min period and used to optimize and validate the rapid immunological diagnostic caries susceptibility assay. The volume and flow rate are recorded and used to normalize all immunological assays. An aliquot of saliva is removed for bacteriology (see below). The remaining saliva is centrifuged at 5,000×g for 10 min and the supernatant heat inactivated at 56° C. for 30 min and either used immediately or frozen at −20° C. for a short period of time (<2 weeks) until needed.

### D. Enumeration of S. mutans in Saliva

An aliquot of each whole saliva is used immediately to quantitate numbers of S. mutans streptococci and is homogenized by vortexing for 20 sec, followed by sonication for 20 sec at a setting of 20 (50 Sonic Dismembrator, Fisher), and finally vortexing again for 20 sec. The number of S. mutans cells is determined by culturing 1:10, 1:100 and 1:1,000 dilutions (double plated) aliquots of each sample, using a Spiral Plater (Spiral Systems) on mitis salivarius agar (Difco Laboratories, Detroit, Mich.) supplemented with 15% sucrose and bacitracin (MSSB; Gold et al., 1973), and incubated at 37° C. in 5% CO₂ and 95% air for 72 h. The numbers of S. mutans streptococci colonies is then enumerated and results reported as the number of S. mutans streptococci/ml of saliva.

### E. Determination of Total Salivary IgA

In order to identify IgA deficient subjects that may affect the rapid diagnostic assay data, the levels of IgA in saliva are determined by a sandwich enzyme-linked immunosorbent assay (ELISA), as follows. Briefly, goat anti-human IgA is used as the coating antibody at 1 μg/ml in 100 μl of 0.1 M carbonate buffer (pH 9.6) and added to wells of flat-bottom polystyrene microtiter plates (EIA, Linbro, Flow Laboratories, Inc., McLean, Va.) as described previously (Gregory et al., 1990). Plates are incubated for 3 h at 37° C. and overnight at 4° C. and the wells are thrice-washed with saline containing 0.05% Tween 20 (Tween-saline) to remove unbound antibody. After the final wash, 200 μl of a blocking solution containing 1% globulin-free human serum albumin (Sigma Chemical Co., St. Louis, Mo.) in carbonate buffer is added and the plates incubated at 25° C. for 60 min. The wells are washed as before and 100 μl of the various samples (at dilutions ranging from 1:10 to 1:100 in Tween-saline) is added to each well and incubated at 37° C. for 60 min. Standards include commercial human colostral IgA. The wells are washed, 100 μl of horseradish peroxidase-labeled goat IgG anti-human IgA (Cappel Scientific Division, Cooper Biomedical, Inc., Malvern, Pa.) is added and the plates incubated at 37° C. for 3 h then overnight at 4° C. The wells are washed and 100 μl of a substrate solution composed of 0.4 mg/ml of orthophenylene diamine HCl (Sigma) dissolved in citrate buffer (pH 5.0) and containing 0.025% H₂O₂ is added to each well and reacted at 25° C. for 30 min until stopped by addition of 100 μl of 2 N H₂SO₄. The amount of color which develops is measured at 490 nm in the microtiter plate by using a ELISA microplate reader (Molecular Devices). Standard curves are generated and the concentrations of salivary IgA will be reported as μg/ml.

### F. Confirmation of Salivary IgA Antibody to S. mutans by ELISA

In order to confirm levels of salivary IgA antibody to S. mutans antigens measured in the rapid diagnostic assay, polystyrene microtiter plates (EIA, Linbro, Flow Laboratories, Inc., McLean, Va.) are coated (100 μl well) with either formaldehyde-killed bacteria (diluted to 0.5 optical density at 540 nm in carbonate/bicarbonate buffer), the enriched-fimbrillar preparation (1 μg/ml diluted in 0.1 M carbonate/bicarbonate buffer, pH 9.6), or other S. mutans antigens (GTF or antigen I/II; 1 μg/ml diluted in 0.1 M carbonate/bicarbonate buffer) and incubated at 37° C. for 3 h. Coated plates are washed three times in Tween saline (TS; 0.9% NaCl containing 0.05% Tween 20) to remove unbound antigen. Free sites on the plates are blocked by reaction with 200 μl of a solution containing 10 μg/ml of human serum albumin (Sigma) for 1 h at 25° C. Diluted saliva samples (diluted 1:10 in TS) are added (100 μl/well) to the wells, in triplicate, and incubated for 2 h at 37° C. Antigen added without saliva, but with TS, will serve as the negative control. A saliva sample from a previously known high antibody producer CF subject will serve as a reference control. The plates are washed three times with TS and incubated for 3 h at 37° C. with 100 μl/well of horseradish peroxidase-labeled anti-human IgA heavy chain specific reagent (Sigma; 1:1000).

After washing three times with TS, orthophenylenediamine dihydrochloride (0.5 mg/ml) in 0.05 M citrate buffer (pH 5.0) containing 0.7 μl of 30% H_{2O}₂/ml of substrate is added (100 μl) to every well. Color development is monitored between 10 and 30 min, and the reaction stopped using 2 N H₂SO₄ (100 μl/well). The amount of color that developed is measured at 490 nm in the microtiter plate with a Molecular Devices spectrophotometer. The background values are automatically subtracted from the experimental samples. The data is reduced by computing the means and standard errors of the mean of the absorbances of triplicate determinations per sample. Correlation of ELISA antibody levels with immunodiagnostic assay results are used to assess the success of the assay.

### G. Troubleshooting

Several problems with the studies described here may develop such as the saline treated strips may develop a blue line at the location of the anti-human IgA band. This most likely would be due to cross-reactivity between the S. mutans antigen and the antibody and can be removed by either dilution or adsorption of the cross-reactive antibodies. This has not been observed in preliminary studies. Alternatively, the strip may not develop a blue line at the appropriate location implying a lack of antibody or insufficient reactivity with the beads. Approaches will include using different sources of saliva that have previously demonstrated high levels of salivary IgA antibody to S. mutans by ELISA and to increase the concentration of the saliva and/or anti-human IgA reagent. However, this also has not been observed in the preliminary experiments.

Problems may also arise as a result of primarily the lack of discrimination of positive and negative results in the assay. For resolution of these problems, the dilution of saliva, concentration and type of S. mutans antigen and concentration of anti-human IgA is adjusted to allow the assay to discriminate between saliva from caries-free and caries-active subjects.

### H. Determination of Levels of Reactivity of Salivary Antibody from Defined Caries-Active and Caries-Free Children in the Optimized Assay

In order to determine the level of reactivity of clinical samples from defined caries-active and caries-free children, saliva is used from the ongoing clinical study described above. Saliva samples are used from Indiana children who are determined to be canes free or active in a blind experiment. Saliva samples are coded by clinical staff so that the identity and caries-status of the patient is unknown. Furthermore, the rapid diagnostic assay will be used on the samples within 2 weeks of collection. The caries status of the subjects will not be known until the re-evaluation 12 months later. In this study, caries active subjects are defined as individuals with five or more carious lesions at project initiation and must develop at least 2 new carious lesions (DMFT) in the previous 12 months. Likewise caries free subjects are defined as individuals who have never had a carious lesion and did not develop a lesion in the same 12 month interval. These children are all the same age (10-13 years old) and from the same city (Connersville, Ind.) which provided for all variables being similar such as water supply, living conditions, and socioeconomic status. Samples are assessed using the rapid diagnostic assay provided herein. Results (presence or absence of a blue band) are correlated with type of saliva assessed (caries-free or caries-active). Statistical analysis is used to establish sensitivity and specificity of the assay. All samples are assessed in duplicate at least 2 times to ensure reproducibility of the assay. Each sample is further assessed for antibody activity by ELISA as previously described (Gregory et al., 1990) to ensure correct placement of samples into groups (i.e., high antibody-caries resistant; low antibody-caries active).

### I. Optimization of the Threshold of Immune Reactivity of Assay

The dilution of saliva from the caries-free and caries-active pediatric population, concentration and type of S. mutans antigen and concentration of anti-human IgA is adjusted to optimize the assay. Saliva dilutions initially were 1:3 and 1:10 but other dilutions will be examined including a range of between 1:3 and 1:100. Optimal saliva dilutions in the ELISA assays were typically in the 1:4 and 1:10 range. The concentration of the autoclaved extract will also be adjusted. Initially 10 μg of extract/100 μg of beads were used with success. Other extract concentrations that may need to be examined are between 1 μg/10 mg and 50 μg/100 μg of beads. Concentrations are carefully examined to allow optimal discrimination between caries-free and caries-active saliva samples. In addition, different antigens are assessed to allow optimal discrimination. Antigens to be examined include S. mutans GTF, fimbriae and sAgI/II in addition to the crude extract. These antigens are prepared using established procedures (fimbriae, GTF or sAgI/II; Fontana et al., 1995; Taubman and Smith, 1976; Russell et al., 1980, respectively). The concentration of anti-human IgA is adjusted to allow optimal discrimination of positive and negative samples. Initially a concentration of 1 μg/ml was used with success but other concentrations between 0.1 and 50 μg/ml is assessed if necessary.

### J. Statistical Analysis

The whole saliva number of S. mutans streptococci/ml of saliva and salivary IgA antibody levels are summarized (mean, standard deviation, range) for the CF and CS groups. Two-sample t-tests are conducted to verify differences in S. mutans between the two groups. All samples are assayed in duplicate and most experiments with the same samples are done at least three times. Reproducibility of the assay is assessed by computing percentage agreement and kappa statistics. Sensitivity, specificity, positive predictive value, negative predictive value, and percentage correct of the assay is assessed using the previously described CF and CS children. 95% confidence intervals are computed for all parameters. The data is further examined to determine if the false positives and false negatives can be identified by their levels of salivary IgA antibody to S. mutans as determined by ELISA. The data from this study is used for sample size calculations for prospective studies addressing the ability of the assay to determine future caries susceptibility.

### K. Human Subjects

Saliva samples from laboratory personnel for optimization of the assay were collected and IRB approval was obtained for this (study #9104-24). Human subjects will be used to determine the threshold between detecting caries free status and caries at risk subjects. Human use approval for the recruitment of the caries-free and caries-active children in Connersville, Ind. is pending.

### IV. PARTICLES AND PARTICLE DETECTION

Certain embodiments of the invention comprise use of microparticles. By “microparticles” it is meant any object capable of diffusing or being suspended in human saliva or a dilution thereof and to which an antigen can be attached. The particles will generally be labeled or colored to facilitate detection of the presence of the particles. By “colored” it will be understood to those of skill in the art that any particular color of particles could be used provided the color can be distinguished from, for example, the solid phase and/or other particles or labels, when appropriate. Thus, a solid support may be colored to provide contrast with the particles and allow visual detection of particles.

Microparticles may be composed of any suitable type of material, including, but not limited to: polystyrene, polymethylacrylate, polypropylene, latex, polytetrafluoroethylene, polyacrylonitrile, and polycarbonate, or similar materials. Examples of materials for use of colored particles include colloidal metals, such as gold and dyed particles as described in U.S. Pat. Nos. 4,313,734 and 4,373,932, the disclosures of which are specifically incorporated herein by reference. In addition to direct coloration of particles, various labels may attached to the particles to facilitate detection of the presence of absence of the particles. For example, labels could be used including, but not limited to: radiolabels, chromophores, fluorophores, chemiluminescent moieties, antigens and transition metals. Preferably, a label will be visually detectable with the naked eye. Alternatively, detection can be facilitated with a charge-coupled device (CCD), fluorescence microscopy, or laser scanning (U.S. Pat. No. 5,445,934, specifically incorporated herein by reference in its entirety). The presence of a label may also be detected using a variety of other techniques, such as an assay with a labeled enzyme, antibody, or the like. Other techniques using various marker systems for detecting bound particles will also be readily apparent to those skilled in the art.

In accordance with the invention, one or more S. mutans antigens can be attached to particles via covalent binding and/or adsorption or other known methods. For example, particles, such as latex particles, can be coated with antigens as is described herein below. Similarly, secreted salivary antibodies to more than one S. mutans antigen can be detected collectively by coating microparticles with a plurality of S. mutans antigens. Alternatively, the antibodies could be detected individually or in various combinations by using differentially labeled or colored microparticles coated with the desired combination or individual antigen(s). In this way, the relative risk of various dental diseases that are associated with the presence or absence of antibodies to a given antigen can be assessed.

Particle size can vary that is used may vary. In initial studies, 0.200 μm (styrene/vinyl carboxylic acid blue, 0.289 μm (styrene/vinyl carboxylic acid blue) and 0.360 μm (styrene/glycidylmethacrylate/epoxy blue) beads from Bangs Laboratories, Inc., Fishers, Ind. were used. These and any many other size beads could be used. In certain embodiments of the invention, the 0.360 μm beads were found to function well. Generally, it will be preferred that the average diameter of the particles be smaller than the average pore size of the support material being used. Thus, embodiments which utilize various other solid phases also are contemplated and are within the scope of this invention.

### V. SOLID SUPPORTS

A solid support, or “solid phase,” used in accordance with the invention can comprise potentially any suitable material with sufficient porosity to allow access to salivary proteins, and specifically, secreted IgA antibodies. The solid support should be a substance that itself does not adsorb IgA molecules to any significant extent and that has a broad range of chemical, physical and thermal stability. Generally, a ligand will be coupled to the solid support, wherein the ligand is capable of binding secretory IgA. The ligand should be coupled in such a way as to not affect its binding properties. The solid support will also comprise microparticles reversibly attached to the support. By “reversibly attached,” it is meant that the microparticles will be capable of migrating along the solid support when contacted with saliva or a dilution thereof. Thus, the reversible attachment may merely comprise depositing the particles on or in the solid support, or may comprise use of a linkage soluble in saliva. The ligand should be relatively tightly bound to the solid phase, preferably remaining bound to the solid support following contact with saliva.

Although microporous structures may be preferred for use as the solid phase support in the assay, materials with gel structure in the hydrated state could be used as well. All that is necessary for a material used as the solid support is that the microparticles can be successfully contacted with the test sample and, subsequently, with the ligand. Examples of such materials include, but are not limited to, for example, natural polymeric carbohydrates and their synthetically modified, crosslinked or substituted derivatives, such as agar, agarose, cross-linked alginic acid, substituted and cross-linked gnar gums, cellulose esters, especially with nitric acid and carboxylic acids, mixed cellulose esters, and cellulose ethers; natural polymers containing nitrogen, such as proteins and derivatives, including cross-linked or modified gelatins; natural hydrocarbon polymers, such as latex and rubber; synthetic polymers which may be prepared with suitably porous structures, such as vinyl polymers, including polyethylene, polypropylene, polystyrene, polyvinylchloride, polyvinylacetate and its partially hydrolyzed derivatives, polyacrylamides, polymethacrylates, copolymers and terpolymers of the above polycondensates, such as polyesters, polyamides, and other polymers, such as polyurethanes or polyepoxides; porous inorganic materials such as sulfates or carbonates of alkaline earth metals and magnesium, including barium sulfate, calcium sulfate, calcium carbonate, silicates of alkali and alkaline earth metals, aluminum and magnesium; and aluminum or silicon oxides or hydrates, such as clays, alumina, talc, kaolin, zeolite, silica gel, or glass; and mixtures or copolymers of the above classes, such as graft copolymers obtained by initializing polymerization of synthetic polymers on a pre-existing natural polymer. Any of these or other materials can be used in suitable shapes, such as films, sheets, or plates, or they may be coated onto or bonded or laminated to appropriate inert carriers, such as paper, glass, plastic films, fabrics or any other such suitable materials. Nitrocellulose will be of particular use for such applications.

It is contemplated that such porous solid supports described herein above are preferably in the form of sheets of thickness from about 0.01 to 0.5 mm, preferably about 0.1 mm. The pore size may vary within wide limits, and is preferably from about 0.025 to 15 microns, especially from about 0.15 to 15 microns. The surfaces of such supports may be activated by chemical processes Which cause covalent linkage of the ligand to the support material.

Preferred solid phase materials for assays, in addition to nitrocellulose, include filter paper such as a porous fiberglass material or other fiber matrix materials. The thickness of such material is not critical and will be a matter of choice, largely based upon the properties of the sample or analyte being assayed, such as the fluidity of the test sample.

### VI. LINKERS/COUPLING AGENTS

In certain embodiments of the invention, a ligand and/or microparticles may be permanently or reversibly bound to a solid phase. Similarly, certain embodiments of the invention may comprise binding one or more antigens or labels to microbeads or antibodies. Numerous techniques are known to those of skill in the art for creating such linkages and may be used with the invention.

In the instant invention, it will generally be desirable that the attachment of microbeads to the solid phase be reversible in saliva solution. That is, that when contacted with saliva or dilutions thereof, that the microbeads be capable of migrating along the solid phase so that they are capable of contacting the ligand. Thus, the attachment should be soluble when in solution. However, the ligand will preferably be non-reversibly bound at one or more selected discrete locations on the solid phase. In this way, labeled microbeads that become bound by the ligand can be detected.

Numerous disulfide-bond containing linkers are know and can successfully be employed to conjugate moieties in accordance with the invention. Any other linking/coupling agents and/or mechanisms known to those of skill in the art can also be used to combine components or agents used with the invention, such as, for example, antibody-antigen interaction, avidin biotin linkages, amide linkages, ester linkages, thioester linkages, ether linkages, thioether linkages, phosphoester linkages, phosphoramide linkages, anhydride linkages, disulfide linkages, ionic and hydrophobic interactions, bispecific antibodies and antibody fragments, or combinations thereof.

Cross-linking reagents may also be used to form molecular bridges that tie together functional groups of two different molecules, e.g., a stablizing and coagulating agent. However, it is contemplated that dimers or multimers of the same analog can be made or that heteromeric complexes comprised of different analogs can be created. To link two different compounds in a step-wise manner, hetero-bifunctional cross-linkers can be used that eliminate unwanted homopolymer formation. Numerous examples of such linkers are known to those of skill in the art and include, for example, those presented in Table 1, below.

TABLE 1
HETERO-BIFUNCTIONAL CROSS-LINKERS

| | | | Spacer Arm |
|---|---|---|---|
| | | | Length\after |
| linker | Reactive Toward | Advantages and Applications | cross-linking |
| SMPT | Primary amines | Greater stability | 11.2 A |
| | Sulfhydryls | | |
| SPDP | Primary amines | Thiolation | 6.8 A |
| | Sulfhydryls | Cleavable cross-linking | |
| LC-SPDP | Primary amines | Extended spacer arm | 15.6 A |
| | Sulfhydryls | | |
| Sulfo-LC-SPDP | Primary amines | Extended spacer arm | 15.6 A |
| | Sulfhydryls | Water-soluble | |
| SMCC | Primary amines | Stable maleimide reactive group | 11.6 A |
| | Sulfhydryls | Enzyme-antibody conjugation | |
| | | Hapten-carrier protein conjugation | |
| Sulfo-SMCC | Primary amines | Stable maleimide reactive group | 11.6 A |
| | Sulfhydryls | Water-soluble | |
| | | Enzyme-antibody conjugation | |
| MBS | Primary amines | Enzyme-antibody conjugation | 9.9 A |
| | Sulfhydryls | Hapten-carrier protein conjugation | |
| Sulfo-MBS | Primary amines | Water-soluble | 9.9 A |
| | Sulfhydryls | | |
| SIAB | Primary amines | Enzyme-antibody conjugation | 10.6 A |
| | Sulfhydryls | | |
| Sulfo-SIAB | Primary amines | Water-soluble | 10.6 A |
| | Sulfhydryls | | |
| SMPB | Primary amines | Extended spacer arm | 14.5 A |
| | Sulfhydryls | Enzyme-antibody conjugation | |
| Sulfo-SMPB | Primary amines | Extended spacer arm | 14.5 A |
| | Sulfhydryls | Water-soluble | |
| EDC/Sulfo-NHS | Primary amines | Hapten-Carrier conjugation | 0 |
| | Carboxyl groups | | |
| ABH | Carbohydrates | Reacts with sugar groups | 11.9 A |
| | Nonselective | | |

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in saliva will be employed when binding an antigen to a microparticle of the ligand to the solid phase. Numerous types of disulfide-bond containing linkers are known that can be successfully employed to achieve such linkages. Linkers that contain a disulfide bond that is sterically hindered may prove to give greater stability in bodily fluids such as saliva, preventing release of the linkage. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Thorpe et al., 1987). The use of such cross-linkers is well understood in the art. Another embodiment involves the use of flexible linkers.

U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Preferred uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

### VII. ANTIBODIES

Antibodies may find use in certain embodiments of the invention. For example, monoclonal or polyclonal antibodies may be used as ligands specific for human secretory IgA in accordance with the invention. Means for preparing and characterizing such antibodies are well known in the art (see, e.g., Antibodies: A Laboratory Manual, Cold Spring Harbor Laboratory, 1988; incorporated herein by reference). The methods for generating monoclonal antibodies (mAbs) generally begin along the same lines as those for preparing polyclonal antibodies. Briefly, a polyclonal antibody is prepared by immunizing an animal with an immunogenic composition in accordance with the present invention and collecting antisera from that immunized animal. A wide range of animal species can be used for the production of antisera. Typically the animal used for production of antisera is a rabbit, a mouse, a rat, a hamster, a guinea pig or a goat. A rabbit is a preferred choice for production of polyclonal antibodies because of the ease of handling, maintenance and relatively large blood volume.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin also can be used as carriers. Means for conjugating a polypeptide to a carrier protein are well known in the art and include glutaraldehyde, m-maleimidobencoyl-N-hydroxysuccinimide ester, carbodimide and bis-biazotized benzidine.

As is also well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection also may be given. The process of boosting and titering is repeated until a suitable titer is achieved. When a desired level of immunogenicity is obtained, the immunized animal can be bled and the serum isolated and stored, and/or the animal can be used to generate mAbs.

Monoclonal antibodies may be readily prepared through use of well-known techniques, such as those exemplified in U.S. Pat. No. 4,196,265, incorporated herein by reference. Typically, this technique involves immunizing a suitable animal with a selected immunogen composition, e.g., purified or partially purified human IgA. The immunizing composition is administered in a manner effective to stimulate antibody producing cells. Rodents such as mice and rats are preferred animals, however, the use of rabbit, sheep, or frog cells also is possible. The use of rats may provide certain advantages (Goding 1986), but mice are preferred, with the BALB/c mouse being most preferred as this is most routinely used and generally gives a higher percentage of stable fusions.

Following immunization, somatic cells with the potential for producing antibodies, specifically B lymphocytes (B cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render them incapable of growing in certain selective media which support the growth of only the desired fused cells (hybridomas).

Any one of a number of myeloma cells may be used, as are known to those of skill in the art (Goding 1986; Campbell 1984). For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions.

One preferred murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line.

Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Sendai virus have been described (Kohleretal., 1975; 1976), and those using polyethylene glycol (PEG), such as 37% (v/v) PEG, (Gefter et al., 1977). The use of electrically induced fusion methods also is appropriate (Goding 1986).

Fusion procedures usually produce viable hybrids at low frequencies, about 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B-cells can operate this pathway, but they have a limited life span in culture and generally die within about two weeks. Therefore, the only cells that can survive in the selective media are those hybrids formed from myeloma and B-cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide mAbs. The cell lines may be exploited for mAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide mAbs in high concentration. The individual cell lines also could be cultured in vitro, where the mAbs are naturally secreted into the culture medium from which they can be readily obtained in high concentrations. mAbs produced by either means may be further purified, if desired, using filtration, centrifugation and various chromatographic methods such as HPLC or affinity chromatography.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Preliminary Studies

Studies were carried out to investigate naturally occurring immune responses to S. mutans in human populations for diagnostic purposes. Preliminary studies were conducted to determine the degree of antigen binding to 3 different latex beads (styrene/vinyl carboxylic acid blue 0.200 μm, styrene/vinyl carboxylic acid blue 0.289 μm and styrene/glycidylmethacrylate/epoxy blue 0.360 μm beads, (Bangs Laboratories, Inc., Fishers, Ind.)). A crude mixture of surface antigens of S. mutans strain A32-2 was prepared by autoclaving the bacterial cells in saline (Rantz and Randall extract, Rantz and Randall, 1955). This preparation has been previously determined to contain most, if not all, S. mutans cell surface antigens including fimbrial, GTF, and sAgI/II components. Studies indicated that the antigens coated each of the 3 beads by testing the coated beads with a 1:10 dilution of saliva followed by examination under a microscope. Saliva agglutinated each of the three antigen-coated beads well but not uncoated beads, indicating that salivary IgA antibody bound to and agglutinated the antigen-coated beads. However, the two carboxylic acid beads had distinctly larger aggregates in the samples without saliva making the movement of the antigen-coated beads through the strip difficult. Therefore, the 0.360 μm epoxy beads were selected for further use.

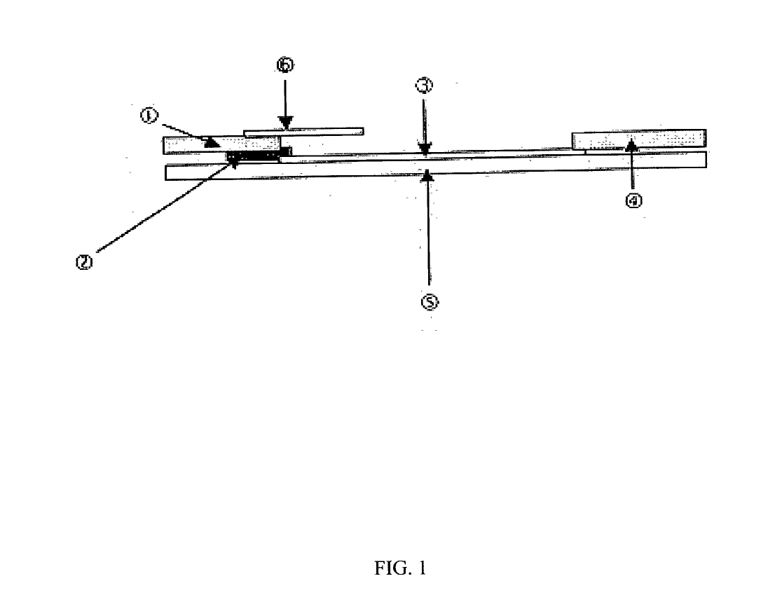

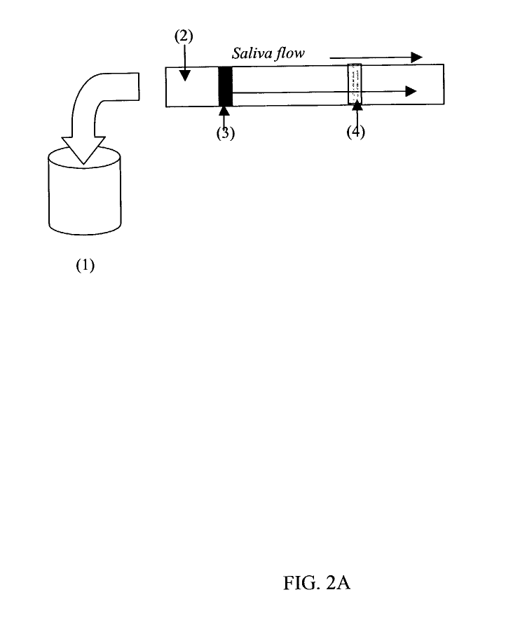

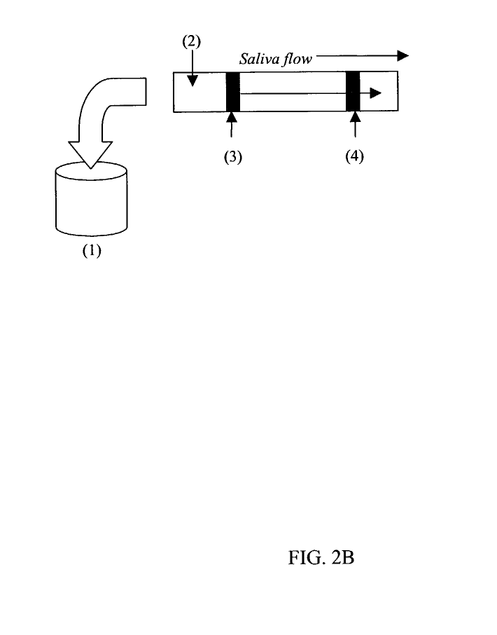

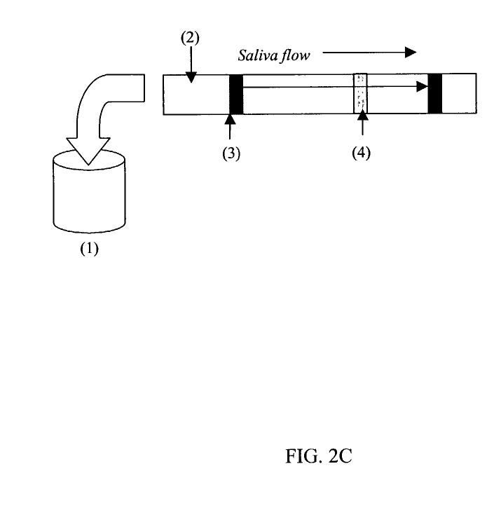

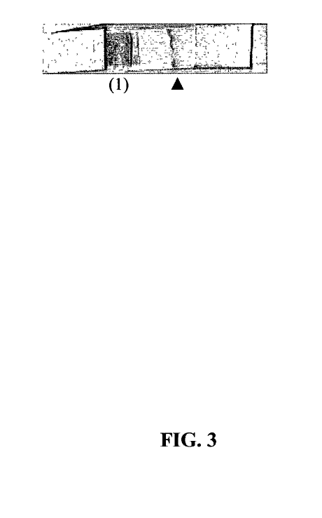

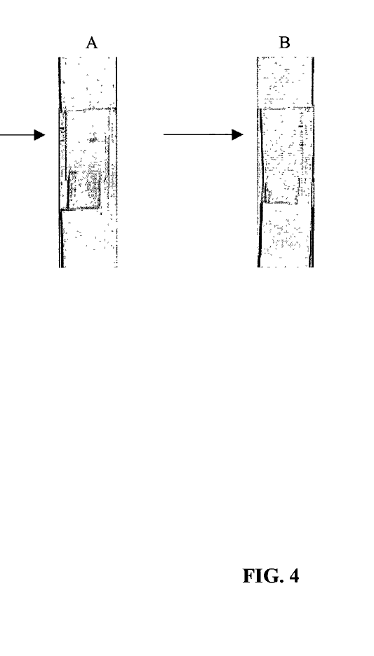

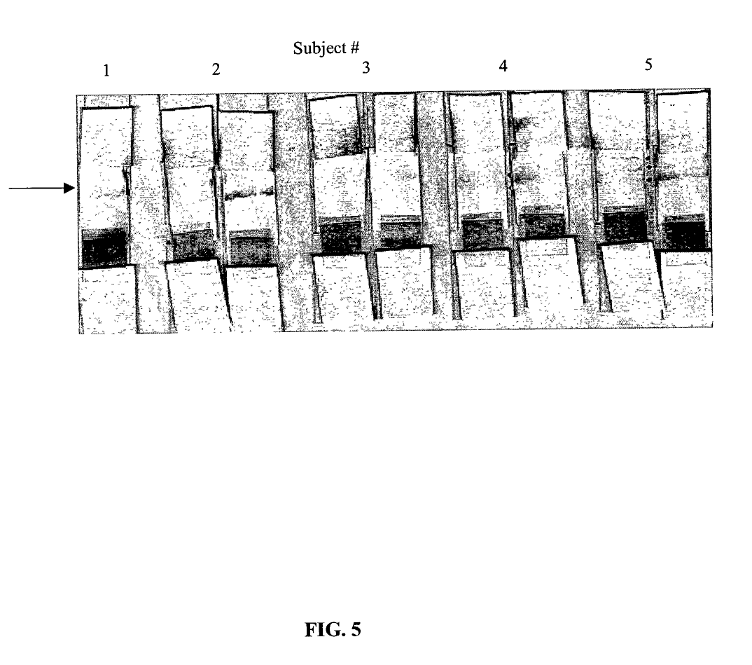

Immunochromatographic test strips (Millipore Corp., Bedford, Mass.) were used to prepare assays using the technology described below (see FIG. 1). Initially, styrene/vinyl carboxylic acid blue 0.200 μm beads were coated with S. mutans antigen and applied to the packaged conjugate pad containing a 60 cm/sec flow rate Hi-Flow membrane to examine the ability of the beads to migrate through the strip after dipping the strip in saline. These antigen-coated beads were unable to migrate possibly because the beads had undergone self-aggregation (see above) causing lack of mobility through the strip so the beads were sonicated. However, the sonicated beads were still unable to migrate through the strip. The use of epoxy beads, however, was found to eliminate this problem, with the new beads able to migrate through the strip properly. Following dipping the strip in a 1:10 dilution of saliva (see FIGS. 2A-C), binding of the antigen-coated beads at the site of the anti-human IgA line was achieved indicating that the assay was functioning properly to identify the presence of salivary IgA antibodies to S. mutans (FIG. 3). A strong band of latex beads coated with S. mutans antigen and salivary IgA antibodies immobilized by anti-human IgA was visible after dipping in saliva. Saline used in place of saliva did not cause the presence of a blue line (FIG. 4, panel A, B). Saliva from a selection of individuals assessed under the stated conditions caused the appearance of a blue line (FIG. 5). Detection was achieved by migration and binding of the blue antigen-coated beads to the anti-human IgA band on the membrane with saliva (1:3 dilution on right and/or 1:10 dilution on left) from 5 normal subjects. Subject 1 only had 1:3 dilution of saliva and subjects 2-5 had both dilutions.

Bands obtained using standard laboratory pipettes were not straight or evenly distributed. This may be corrected by the use of a laser application process to allow the use of narrow straight lines of anti-human IgA and latex beads resulting in tight discrete bands. Additional studies are being conducted to further dissociate microaggregates of any type of bead that is used that may be causing retention of the coated beads on the strip. In addition, other buffers and blocking solutions are currently being assessed in this process.

### Example 2

### Assay Preparation and Use

### A. Harvesting S. mutans Cell Surface Antigens

The procedure is as follows. On day 1, S. mutans strain A32-2 is inoculated from frozen stock into tube #1 containing 7 ml broth. On day 2, streak S. mutans on plate containing TSA to check for purity. On day 3, verify purity of S. mutans, transferring 1 loop to tube #2, which also contains 7 ml broth. On day 4, pour entire tube #2 into 500 ml flask of TSB, BHI. On day 5, harvest S. mutans by centrifugation, wash twice in saline, resuspend in 100 ml saline, autoclave cells and centrifuge. The supernatant is saved and frozen in aliquots.

### B. Procedure for Adhering S. mutans Antigen to the Carboxyl-Modified Microspheres

Wash 200 μl of microspheres twice in 2 ml of Activation Buffer (Acetate Buffer—0.1M Acetic acid, 0.1M Sodium acetate, pH 4.8). Washing is accomplished by centrifuging at 12,000 rpm for 15 min. After second wash, resuspend pellet in 2 ml of activation buffer. While mixing, add 20 mg of 1-Ethyl-3-(3-dimethylaminopropyl) carbodiimide. Allow to react for 15 min at room temperature with continuous mixing. Wash twice in Coupling Buffer (PBS), completely resuspend in 1 ml of the same. Dissolve S. mutans antigen in 1 ml of PBS and combine with microspheres. React at room temperature for 2-4 h with constant mixing. Wash, resuspend in 2 ml of quenching solution (PBS with 30 mM glycine and 1% BSA), mix gently for 30 min. Wash, resuspend in 2 ml storage buffer (PBS and 0.05% BSA and 0.1% Sodium Azide). Store at 4° C. until used.

### C. Procedure for Adhering S. mutans antigen to the Epoxy-Modified Microspheres:

Wash 200 μl of microspheres twice in 2 ml of Wash Buffer (1.0M NaCl). Washing was accomplished by centrifuging at 1,000 rpm for 5 min. After second wash, resuspend pellet in 1 ml of Coupling Buffer (Carbonate-Bicarbonate—0.1M Sodium carbonate, 0.1M Sodium bicarbonate, pH 9.8). Combine microsphere solution with 1 ml S. mutans antigen solution. Allow to react 24-48 h at room temperature. Wash, resuspend in 2 ml of quenching solution (1.0M NaCl and 1% BSA), mix gently for 30 min. Wash, resuspend in 2 ml of storage buffer (PBS and 0.05% BSA and 0.1% Sodium Azide). Store at 4° C. until used.

### D. Procedure for Preparing Assay Strip and Conducting the Assay

A rectangular sheet of 3-10 micron pore size nitrocellulose membrane is cut with dimensions of 15 cm×8 cm. Place membrane and conjugate pad onto laminate card (see FIG. 1). Apply 10% sucrose solution to nitrocellulose membrane and conjugate pad. Allow to dry for 1 h at room temperature. Anti-human IgA (Fc specific, diluted 1:2) is applied with a micropipet to the strip approximately 3 cm from the top of the strip. The width of this antibody should be approximately 2 mm. Allow to dry for 1 h at room temperature. Add sample pad and absorbent pad to strip. Sonicate antigen-coated beads for 20 sec, and bring to 1% Triton and 0.05% BSA. Apply antigen-coated beads to the conjugate pad which was previously coated with a sucrose glaze. Apply the top laminate over the sample pad/conjugate pad/membrane as shown in FIG. 1. Dip the sample pad into the saliva solution and observe for the presence of a blue band at the location of the anti-human IgA line after 3-4 min.

### E. Results

The results indicated that when the strip was dipped in saliva, the saliva moved up the paper by capillary action. When the saliva reached the latex beads coated with S. mutans antigen, any S. mutans-specific secretory IgA binds to the beads. The beads move up the paper where they meet a fixed band of anti-human IgA. An antigen-antibody reaction occurs immobilizing beads containing salivary secretory IgA anti-S. mutans antibodies, thus causing a blue line to develop on the nitrocellulose membrane at that location. In contrast, when the strip was dipped in water or in saliva without IgA anti-S. mutans antibodies, the beads moved up the membrane but were not bound by the anti-human IgA. It was discovered that this reaction was optimized by diluting the saliva sample 1:3 and the anti-human IgA 1:2. When using the 1:3 dilution of saliva, it takes the front of beads approximately 120 sec to travel the 4.0 cm to the anti-human IgA location (Table 2).

TABLE 2
Demonstrates length of time (in seconds) required for the solvent
front to travel 4.0 cm using different dilutions of saliva or water.
Saliva Dilution

| | Pure H2O | 1:16 | 1:8 | 1:4 | 1:3 | 1:2 |
|---|---|---|---|---|---|---|
| Trial 1 | 38 | 45 | 97 | 120 | 122 | 126 |
| Trial 2 | 36 | 43 | 90 | 110 | 118 | 120 |
| Average | 37 | 44 | 93.5 | 115 | 120 | 123 |

### Example 3

### Materials and Methods

The materials used included nitrocellulose membranes supplied by Schleicher & Schuell (Keene, N.H.), two different types of blue latex bead particles: carboxyl modified (two sizes: 0.2 μm and 0.289 μm) and epoxy modified (size 0.360 μm) (Bangs Laboratories, Fishers, Ind.), S. mutans antigen (cell wall extract, GTF, fimbriae, or antigen I/II), anti-human IgA (Fc specific; Sigma Chemical Co., St. Louis, Mo.), and saliva samples from individuals known to be caries resistant or caries susceptible.

The first step in preparing an assay was harvesting S. mutans antigen and coating blue latex bead particles with it. Next, the blue latex beads were pipetted onto the nitrocellulose membrane in a straight line approximately one inch from the end that was to be dipped into the saliva. Tests were conducted to develop efficient movement of the beads through the conjugate pad onto the nitrocellulose membrane. In addition, studies were conducted to determine the dilution of saliva which migrated optimally through the strip. After optimizing movement of the beads on the membrane, anti-human IgA (Fc specific) was pipetted in a straight line onto other strips approximately one inch from the opposite. After these strips were prepared, they were dipped in optimally diluted saliva samples to evaluate whether or not the beads containing S. mutans antigen would bind to the line of anti-human IgA (FIG. 2).

### Example 4

### Optimization of Assays

The immunochromatographic test strip prepared as described above will be optimized to react with saliva from laboratory volunteers. The basic parameters of the test strip are described below. Those of skill in the art will recognize that various parameters may be adjusted to obtain optimum reactivity, such as: 1) type and concentration of antigen coating the beads; 2) the size and chemistry of the beads; 3) the buffers and blocking proteins used in the bead preparation and contained on the sample and conjugate pads; 4) the dilution of saliva and buffer diluent; 5) the length of the strip components; 6) the concentration and type of anti-human IgA; and 7) the distance of the anti-IgA trapping line from the bead line on the strip. All parameters may be studied sequentially to enable the optimization of the assay.

Initially, 1:3 and 1:10 dilutions of saliva in saline have been used successfully. Controls include saline in place of saliva. In addition, secretory IgA (Sigma) can be used to coat latex beads at different concentrations and used in the assay in place of the S. mutans antigen coated beads for use as a positive control. This also allows the determination of the minimum amount of salivary IgA antibody to react in this assay and greatly increases the ability to titrate the system to allow the discrimination of caries-free and caries-active subjects. Second, the concentration of S. mutans antigen and which S. mutans antigen works the best should be optimized. This can be begun by using a crude cell wall extract at 10 μg absorbed onto 100 μg of beads, followed by individual antigens such as fimbriae, GTF or sAgI/II prepared using established procedures (Fontana et al., 1995; Taubman and Smith, 1976; Russell et al., 1980). The last variable that needs to be adjusted is the concentration of anti-human IgA. Initially, a 1 μg/ml concentration of anti-human IgA Fc-specific has been used. An Fc-specific reagent is used to ensure that the antigen-binding portion of the antibody is available to react with salivary IgA antibody bound to the antigen-coated beads. These parameters are assessed sequentially following the basic protocol described above.

### Example 5

Procedure for Attaching S. mutans Antigen to the Latex Beads

The latex beads are diluted to 1% (10 mg/ml) with adsorption buffer, a low ionic strength buffer of pH at or near the pI of the coating antigen protein. An appropriate amount of purified ligand (S. mutans antigen) is diluted in the adsorption buffer. The latex bead suspension is added to the appropriate volume of dissolved S. mutans antigen and mixed gently for 1-2 h. The suspension is incubated overnight at 4° C. with constant mixing. The suspension is centrifuged, the supernatant removed, and the latex bead pellet resuspended in storage buffer to the desired storage concentration (10 mg/ml).

The procedure for assembling an immunochromatographic test strip (see FIG. 1) is as follows. A laminate card (Millipore) is cut to size (approximately 1×7 cm) and the covering on the adhesive removed. A strip of Hi-Flow membrane (1×4 cm; Millipore) is placed on the adhesive side of the laminate card. A conjugate pad is placed on the laminate card partially overlaying the membrane. The conjugate pad is soaked with an aqueous solution of inert compound (i.e. bovine serum albumin) or polymer to block excess binding sites for 30 min at room temperature. The blocked conjugate pad is rinsed in distilled water and dried for 30 min at 30° C. A sample pad is placed on the laminate card partially overlaying the conjugate pad and covering the latex bead line. An absorbent pad is placed on the laminate card partially overlaying the membrane. Anti-human IgA (Fc specific) is applied with a micropipet to the membrane approximately 1 cm from the top of the strip. The width of this antibody line should be approximately 0.8 cm. The top laminate is applied partially covering the sample and conjugate pads. The entire assembly is laminated.

The procedure for applying latex beads is as follows: A solution of 30% sucrose in distilled water is prepared and applied to the conjugate pad where the latex beads are to be located (approximately 1 cm from the bottom, with a width of 0.8 cm). The conjugate pad is baked for 1 h at 40° C. The antigen-coated beads are applied to the membrane over the sucrose glaze, keeping dimensions consistent with the sucrose glaze.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. 4,196,265

U.S. Pat. No. 4,317,734

U.S. Pat. No. 4,373,932

U.S. Pat. No. 4,680,338

U.S. Pat. No. 5,141,648

U.S. Pat. No. 5,445,934

U.S. Pat. No. 5,563,250

U.S. Pat. No. 5,856,456

U.S. Pat. No. 5,880,270

Aaltonen et al., Arch. Oral Biol., 32:55-60, 1987.

Antibodies: A Laboratory Manual, Cold Spring Harbor Laboratory, Cold Spring Harbor Press, Cold Spring Harbor, N.Y., 1988.

Ayakawa et al., Infect. Immun., 55:2759-2767.

Bammann and Gibbons, J. Clin. Microbiol., 10:538-543, 1979.

Bang's Laboratories, TechNote #204 Adsorption to Microspheres, TechNote #303 Lateral Flow Tests, 1999.

Bellanti et al., J. Immunol., 94:344-351, 1965.

Bowen, Caries. Res., 3:227-237, 1969.

Camling and Kohler, Arch. Oral Biol., 32:817-823, 1987.

Camling et al., Arch. Oral Biol., 32:21-25, 1987.

Campbell et al., J. Mol Biol., 180:1-19, 1984.

Challacombe and Lehner, J. Dent. Res., 55:C139-C148, 1976.

Clark, Br. J. Exp. Path., 5:141-147, 1924.

Czerkinsky et al., Proc. Natl. Acad. Sci. USA, 84:2449-2453, 1987.

Fitzgerald and Keyes, J. Am. Dent. Assoc., 61:6-61, 1960.

Fontana, Clinical Diag. Lab. Immunol., 2:719-725, 1995.

Forester et al., J. Gen. Microbiol., 129:2779-2788, 1983.

Gahnberg et al., Arch. Oral Biol., 30:551-556, 1985.

Gefter et al., Somatic Cell Genet, 3(2):231-6, 1977.

Germaine and Tellefson, Infect. Immun., 31:598-607, 1981.

Gibbons and Fitzgerald, J. Bacteriol., 98:341-346, 1969.

Gibbons and van Houte, In: Bacterial adherence, Beachey Eed.), Chapman and Hall, NY, 61-104, 1980.

Goding, In: Monoclonal Antibodies: Principles and Practice, 2d ed., Orlando, Fla., Academic Press, pp. 60-61, 65-66, 71-74, 1986.

Gold et al., Archs. Oral Biol., 18:1357-1364, 1973.

Gregory and Filler, Infect. Immun., 55:2409-2415, 1987.

Gregory et al., In: Advances in Mucosal Immunology, Mestecky et al., (eds.), Proc. 7^{th} Intl. Congress Mucosal Immun., Czechoslovakia, 1149-1152, 1995.

Gregory et al., Infect. Immun., 45:286-289, 1984.

Gregory et al., Infect. Immun., 54:780-786, 1986.

Gregory et al., Oral Microbiol Immunol., 5:181-188, 1990.

Gregory et al., Oral Microbiol Immunol., 5:181-188, 1990.

Gregory, Compendium, 15(10):1282-1294, 1994a

Gregory, Lab. Med., 25(11):724-728, 1994b.

Hamada et al., (eds.), In: Molecular Microbiology and Immunobiology of Streptococcus mutans, Elsevier, Amsterdam, 1986.

Hamada et al., Microbiol. Immunol., 22:301-314, 178.

Koga et al., Infect. Immun., 58:289-296, 1990.

Kohler and Milstein, Eur. J. Immunol., 6:511-519, 1976.

Kohler and Milstein, Nature, 256:495-497, 1975.

Lee et al., Infect. Immun., 57:3306-13, 1989.

Lehner et al., Arch. Oral Biol., 23:1061-1067, 1978.

Levine et al., Infect. Immun., 19:107-115, 1978.

Loesche and Straffon, Infect. Immun., 26: 498-507, 1979.

Loesche, Microbiol. Rev., 50:353-380, 1986.

McBride et al., Infect. Immun., 44:68-75, 1984.

McGhee and Mestecky, Ann. NY Acad. Sci., 409:1-896, 1983.

McGhee and Michalek, Ann. Rev. Microbiol., 35:595-638, 1981.

McGhee et al., J. Immunol., 114:300-305, 1975.

Mestecky et al., Clin. Immunol. Immunopathol., 40:105-114, 1986.

Mestecky et al., J. Clin. Invest., 61:731-737, 1978.

Mestecky, Monogr. Allergy, 24:83, 1988.

Michalek et al., Infect. Immun., 12: 69-75, 1975.

Michalek et al., Science, 192: 1238-1240, 1976.

Perrone et al., Clin. Diagn. Lab. Immunol, 4:291-296, 1997.

Pierce et al., Infect. Immun., 37:687-694, 1982.

Radike et al., J. Amer. Dental Assoc., 86:404-8, 1973.

Rantz and Randall, Stanford Med. Bull., 13:290-291, 1955.

Ray et al., Clin. Diagn. Lab. Immunol., 6:400-404., 1998

Rolla, Swed. Dent. J., 1:241, 1977.

Rose et al., Pediatric Dent., 16:272-275, 1994.

Russell et al., Infect. Immun., 28:486-493, 1980.

Singer and Plotz, Am. J. Med., 21:888, 1956.

Smith et al., Oral Microbiol. Immunol., 5:57-62, 1990.

Taubman and Smith, J. Immunol., 118: 710-716, 1976.

Thorpe et al., Cancer Res., 47:5924-31, 1987.

Walker et al., Science, 177:608-610, 1972.

Zanders and Lehner, J. Gen. Microbiol., 122:217-225, 1981.

What is claimed is:

1. A diagnostic apparatus comprising:
(a) a porous solid support;
(b) microparticles reversibly attached to said support, wherein said microparticles are bound to at least a first antigen from Streptococcus mutans and wherein the microparticles are capable of migrating along the support when contacted with human saliva; and
(c) a ligand bound to said support, wherein said ligand has an affinity for at least a first human IgA antibody.

2. The apparatus of claim 1, wherein the solid support comprises nitrocellulose.

3. The apparatus of claim 1, wherein the microparticles are epoxy modified.

4. The apparatus of claim 1, wherein the microparticles are bound to a plurality of antigens from Streptococcus mutans.

5. The apparatus of claim 1, wherein the microparticles are colored.

6. The apparatus of claim 1, wherein the microparticles are labeled.

7. The apparatus of claim 6, wherein the microparticles are labeled with a visually detectable label.

8. The apparatus of claim 7, wherein the microparticles are fluorescently labeled.

9. The apparatus of claim 6, wherein the microparticles are labeled with a second antigen.

10. The apparatus of claim 6, wherein the microparticles are labeled with an enzyme.

11. The apparatus of claim 1, wherein the microparticles comprise latex beads.

12. The apparatus of claim 11, wherein the latex beads average from about 0.15 μm to about 0.3 μm in diameter.

13. The apparatus of claim 1, wherein the microparticles are reversibly bound at a first selected location on said solid support and wherein the ligand is bound at a second location on said solid support.

14. The apparatus of claim 1, wherein said saliva is diluted.

15. The apparatus of claim 14, wherein the saliva is diluted from about 1:2 to about 1:100 in water.

16. The apparatus of claim 14, wherein the saliva is a 1:2 dilution of saliva with water.

17. The apparatus of claim 1, wherein the saliva is a 1:3 dilution of saliva with water.

18. The apparatus of claim 1, wherein the antigen is glucosyltransferase.

19. The apparatus of claim 1, wherein the antigen is Antigen I/II.

20. The apparatus of claim 1, wherein the antigen comprises a fimbrial protein.

21. The apparatus of claim 20, wherein the fimbrial protein is SmaA.

22. The apparatus of claim 1, wherein the ligand is a protein.

23. The apparatus of claim 1, wherein the ligand is an antibody or fragment thereof.

24. The apparatus of claim 23, wherein the antibody binds specifically to a human IgA Fc region.

25. The apparatus of claim 1, further comprising a second ligand bound to said support.

26. The apparatus of claim 25, wherein said second ligand has an affinity for a protein present in said saliva.

27. The apparatus of claim 26, wherein said protein is amylase.

28. The apparatus of claim 25, wherein said second ligand is an antibody.

29. The apparatus of claim 1, wherein said second ligand has an affinity for at least a first human IgM antibody.

30. The apparatus of claim 25, wherein said second ligand is an antibody.

31. The apparatus of claim 25, wherein said second ligand has an affinity for said first antigen from Streptococcus mutans.

32. A method of assaying a patient for susceptibility to caries development comprising the steps of:
(a) obtaining an assay apparatus comprising:
(1) a porous solid support;
(2) microparticles reversibly attached to said support, wherein said microparticles are bound to at least a first antigen from Streptococcus mutans and wherein the microparticles are capable of migrating along the support when contacted with a solution comprising human saliva; and
(3) a ligand bound to said support at a selected location, wherein said ligand has an affinity for a human IgA antibody;
(b) contacting said assay apparatus with saliva from a patient, wherein said saliva is allowed to contact said microparticles and said ligand; and
(c) detecting the presence or absence of microparticles bound to said ligand at said selected location.

33. The method of claim 32, wherein the solid support comprises nitrocellulose.

34. The method of claim 32, wherein the microparticles comprise latex beads

35. The method of claim 32, wherein the microparticles are epoxy modified.

36. The method of claim 32, wherein the microparticles are bound to a plurality of antigens from Streptococcus mutans.

37. The method of claim 32, wherein the microparticles are colored.

38. The method of claim 32, wherein the microparticles are labeled.

39. The method of claim 38, wherein the microparticles are labeled with a visually detectable label.

40. The method of claim 39, wherein the microparticles are fluorescently labeled.

41. The method of claim 38, wherein the microparticles are labeled with a second antigen.

42. The method of claim 38, wherein the microparticles are labeled with an enzyme.

43. The method of claim 32, wherein the microparticles comprise latex beads.

44. The method of claim 43, wherein the latex beads average from about 0.15 μm to about 0.3 μm in diameter.

45. The method of claim 32, wherein the microparticles are reversibly bound at a first selected location on said solid support and wherein the ligand is bound at a second location on said solid support.

46. The method of claim 32, wherein the solution comprising human saliva is diluted.

47. The method of claim 46, wherein the saliva comprises a dilution of from about 1:2 to about 1:100 in water.

48. The method of claim 46, wherein the solution comprising human saliva is a 1:2 dilution of saliva with water.

49. The method of claim 46, wherein the solution comprising human saliva is a 1:3 dilution of saliva with water.

50. The method of claim 32, wherein the antigen is glucosyltransferase.

51. The method of claim 32, wherein the antigen is Antigen I/II.

52. The method of claim 32, wherein the antigen comprises a fimbrial protein.

53. The method of claim 52, wherein the fimbrial protein is SmaA.

54. The method of claim 32, wherein the ligand is a protein.

55. The method of claim 32, wherein the ligand is an antibody or fragment thereof.

56. The method of claim 55, wherein the antibody binds specifically to a human IgA Fc region.

57. A method of dental care comprising:
(a) obtaining saliva from a patient;
(b) rapidly assaying said saliva for an IgA antibody specific to an antigen from Streptococcus mutans while the patient waits, wherein results from said assaying are obtained within 30 minutes from said step of obtaining; and
(c) initiating a course of treatment for prevention of caries development based on said rapid assaying.

58. The method of claim 57, wherein said rapidly assaying comprises the steps of:
(a) obtaining an assay apparatus comprising:
(1) a porous solid support;
(2) microparticles reversibly attached to said support, wherein said microparticles are bound to at least a first antigen from Streptococcus mutans and wherein the microparticles are capable of migrating along the support when contacted with a solution comprising human saliva; and
(3) a ligand bound to said support at a selected location, wherein said ligand has an affinity for a human IgA antibody;
(b) contacting said assay apparatus with saliva from a patient, wherein said saliva is allowed to contact said microparticles and said ligand; and
(c) detecting the presence or absence of microparticles bound to said ligand at said selected location.

59. The method of claim 57, wherein the course of treatment comprises a preventive measure selected from the group consisting of: recommending immaculate oral hygiene, recommending diet modification, applying a sealant, applying an antimicrobial agents, applying a topical fluoride treatments and applying a fluoride varnish.
